(19) United States
(12) Patent Application Publication
(10) Pub. No.: US 2018/0296132 A1
(43) Pub. Date: Oct. 18, 2018

(54) SENSOR DEVICE AND SENSOR CONTROL DEVICE

(71) Applicant: FUJITSU LIMITED, Kawasaki-shi (JP)

(72) Inventor: Takayuki Yamaji, Yokohama (JP)

(73) Assignee: FUJITSU LIMITED, Kawasaki-shi (JP)

(21) Appl. No.: 16/014,288

(22) Filed: Jun. 21, 2018

Related U.S. Application Data
(63) Continuation of application No. PCT/JP2016/052173, filed on Jan. 26, 2016.

(51) Int. Cl. A61B 5/11; A61B 5/0205; A61B 5/024; A61B 5/00

(52) U.S. Cl. CPC A61B 5/1118; A61B 5/0205; A61B 5/0022; A61B 5/7235; A61B 2562/0219; A61B 5/024

(57) ABSTRACT

A sensor device includes: an inertia sensor; an electric wave sensor; and a controller configured to control transmission power of an electric wave of the electric wave sensor in accordance with a gravity direction which is detected by the inertia sensor.

### CROSS-REFERENCE TO RELATED APPLICATION

This application is a continuation application of International Application PCT/JP2016/052173 filed on Jan. 26, 2016 and designated the U.S., the entire contents of which are incorporated herein by reference.

### FIELD

The techniques described herein relate to a sensor device, a sensor control device, a sensor data processing device, a sensor control program, and a sensor data processing program.

### BACKGROUND

There has been a technique which uses an electric wave sensor such as a Doppler sensor to detect vital information such as body movement, heartbeat, respiration of a person.

Related art is disclosed in Japanese Laid-open Patent Publication No. 2015-27008, Japanese Laid-open Patent Publication No. 2007-122433, or Japanese Laid-open Patent Publication No. 2014-039666.

### SUMMARY

According to one aspect of the embodiments, a sensor device includes: an inertia sensor; an electric wave sensor; and a controller configured to control transmission power of an electric wave of the electric wave sensor in accordance with a gravity direction which is detected by the inertia sensor.

The object and advantages of the invention will be realized and attained by means of the elements and combinations particularly pointed out in the claims.

It is to be understood that both the foregoing general description and the following detailed description are exemplary and explanatory and are not restrictive of the invention, as claimed.

### BRIEF DESCRIPTION OF DRAWINGS

FIG. 1 is a block diagram that illustrates a configuration example of a sensor system according to one embodiment.

FIG. 2 is a diagram that illustrates one example of a sensor mounting position (bed installation) according to one embodiment.

FIG. 3 is a diagram that illustrates one example of the sensor mounting position (bed installation) according to one embodiment.

FIG. 4 is a block diagram that illustrates a configuration example of a sensor which is exemplified in FIG. 1.

FIG. 5 is a block diagram that illustrates a configuration example of the sensor which is exemplified in FIG. 1.

FIG. 6 is a block diagram that illustrates a configuration example of an information processing device which is exemplified in FIG. 1.

FIG. 7A is a diagram for explaining one example of the relationship between the gravity direction and an electric wave irradiation surface in a case where the sensor according to one embodiment is installed in a ceiling in an interior space.

FIG. 7B is a diagram for explaining one example of the relationship between the gravity direction and the electric wave irradiation surface in a case where the sensor according to one embodiment is installed in a bed.

FIG. 8 is a flowchart for explaining an action example (first example) of the sensor system exemplified in FIG. 1.

FIG. 9 is a diagram that illustrates one example of an angle table which is used for assessing a sensor installation angle according to one embodiment.

FIG. 10A is a diagram that schematically illustrates a condition in which the sensor is installed in a wall such that the electric wave irradiation surface of the sensor according to one embodiment is directed in an obliquely downward direction from an upper right side of the wall toward the center of the interior space.

FIG. 10B is a diagram that schematically illustrates a condition in which the sensor is installed in the wall such that the electric wave irradiation surface of the sensor according to one embodiment is directed in an obliquely downward direction from an upper left side of the wall toward the center of the interior space.

FIG. 11 is a diagram that illustrates one example of a transmission power control table according to one embodiment.

FIG. 12 is a diagram that illustrates one example of the time change in the body movement amount which is obtained based on an electric wave sensor value of the sensor according to one embodiment.

FIG. 13 is a schematic diagram for explaining the concept of an extended wavelength according to one embodiment.

FIG. 14 is a diagram for explaining a calculation example of the extended wavelength according to one embodiment.

FIG. 15A to FIG. 15C are waveform diagrams for explaining one example of an extended wavelength calculation process.

FIG. 16 is a diagram for explaining another calculation example of the extended wavelength according to one embodiment.

FIG. 17A to FIG. 17D are schematic diagrams for explaining angle information which is calculated based on an inertia sensor value.

FIG. 18 is a flowchart for explaining an action example (second example) of the sensor system exemplified in FIG. 1.

FIG. 19 is a flowchart for explaining an action example (third example) of the sensor system exemplified in FIG. 1.

FIG. 20 is a diagram for explaining threshold value correction which is exemplified in FIG. 19.

### DESCRIPTION OF EMBODIMENT

In a case where an electric wave sensor is installed (may also be referred to as “arranged”) in a space such as an indoor or interior place, the distance between the electric wave sensor and a sensing target may be different in accordance with the installation place. An electric wave that is transmitted by the electric wave sensor has a propagation characteristic of being attenuated as the propagation distance becomes longer.

Thus, in a case where the distance between the electric wave sensor and the sensing target is different, the difference corresponding to the difference in the distance may occur to a detection value of the electric wave sensor.

For example, between a case of installation of the electric wave sensor in an upper portion of the space such as a ceiling and a case of installation in a lower portion of the space such as a bed, the distance from the electric wave sensor to the sensing target that is present in the space tends to be longer in a case of installation in the upper portion in the space than a case of installation in the lower portion of the space. Thus, a detection value is likely to become smaller.

Thus, an error may occur in detection of the body movement, heartbeat, respiration, or the like unless the detection value of the electric wave sensor is corrected in accordance with the installation place of the electric wave sensor.

Detection precision that corresponds to an arrangement position of a sensor unit may be improved.

An embodiment will hereinafter be described with reference to drawings. However, the embodiment described in the following is a merely example and is not intended to exclude applications of various modifications and techniques, which are not explicitly described in the following. Further, various kinds of exemplary modes that will be described in the following may be carried out by properly combining those. Note that in the drawings used in the following embodiment, portions provided with the same reference characters will denote the same or similar portions unless otherwise mentioned.

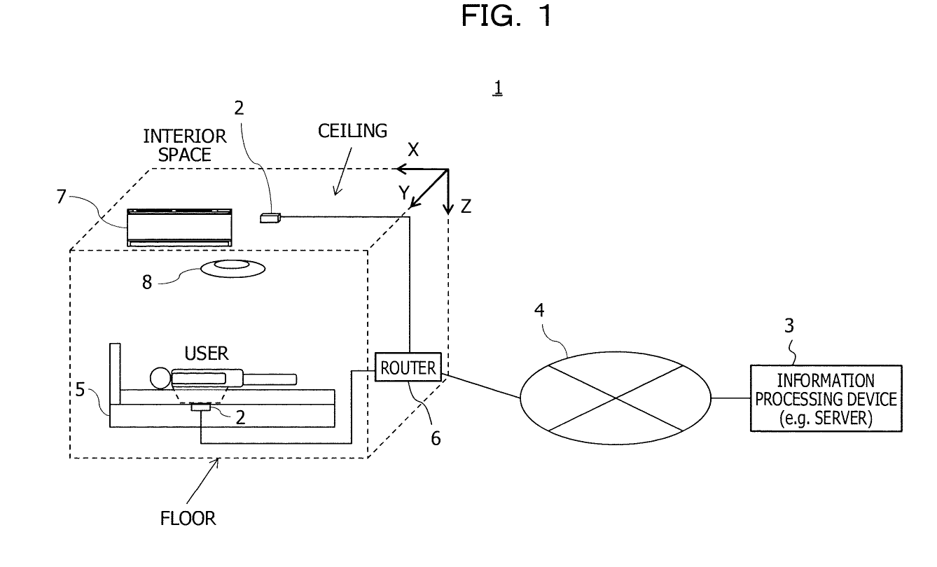

FIG. 1 is a block diagram that illustrates a configuration example of a sensor system according to one embodiment. The sensor system 1 illustrated in FIG. 1 may exemplarily include a sensor 2 and an information processing device 3.

The sensor 2 may exemplarily be connected with a network 4 via a router 6 so as to be capable of communication. Further, the information processing device 3 may be connected with the network 4 so as to be capable of communication. Consequently, the sensor 2 may exemplarily be capable of communication with the information processing device 3 via the router 6 and the network 4. The communication between the sensor 2 and the information processing device 3 may be bidirectional communication.

The sensor 2 may exemplarily transmit information that is obtained by the sensor 2 to the information processing device 3 by communication with the information processing device 3 and receive a control signal for the sensor 2 from the information processing device 3. Information obtained by the sensor 2 may also be referred to as “sensor information”, “sensor data”, or “detection information” for convenience.

The connection between the sensor 2 and the router 6 may be wired connection or wireless connection. In other words, the sensor 2 may include a communication interface (IF) that supports communication by either one or both of wired and wireless connection. For the wireless connection, “Wireless Fidelity (WiFi)”® or “Bluetooth”® may exemplarily be used.

The network 4 may exemplarily be correspondent to a wide area network (WAN), a local area network (LAN), or the Internet. Further, the network 4 may include a wireless access network. For example, the router 6 may be capable of communication with the information processing device 3 by connecting with the wireless access network by a wireless IF.

As already described, the information processing device 3 is capable of communication with the sensor 2 via the network 4 and the router 6. For example, the information processing device 3 may control an action of the sensor 2 based on the information received from the sensor 2 and control an environment of a space that includes a portion or whole of a sensing range of the sensor 2. The space may exemplarily be an indoor (or interior) space. As one non-limiting example, the sensor 2 may be installed (may also be referred to as “arranged”) in an interior space such as a bedroom.

Control of the action of the sensor 2 may exemplarily include control of transmission power of an electric wave that is transmitted by the sensor 2 as described later. Thus, the information processing device 3 may also be considered as one example of “sensor control device”. Further, control of the environment in the interior space may exemplarily include controlling the interior space to a comfortable environment for a user in the interior space.

For example, the information processing device 3 controls, for example, the temperature, air volume, and air direction of an air conditioner 7 and dimming of lighting equipment 8, which are installed in the interior space, based on the sensor information by communication via the network 4 and may thereby control the interior space to a comfortable environment for the user. The control may exemplarily be control that assists a good sleep of the user. Such control may also be referred to as “good sleep control” for convenience.

In order to enable control of an interior environment, similarly to the sensor 2, the air conditioner 7 or the lighting equipment 8 may exemplarily be connected with the router 6 by wired or wireless connection so as to be capable of communication and may be capable of communication with the information processing device 3 via the router 6 and the network 4.

The information processing device 3 may exemplarily be configured by using one or plural servers. In other words, the sensor 2 or the environment of the interior space may be controlled by one server or may be controlled by distributed control by plural servers. The server may be correspondent to a cloud server that is included in a cloud data center, for example.

The air conditioner 7 or the lighting equipment 8 may be for either one of household use and business use. The air conditioner 7 or the lighting equipment 8 for household use is one example of a so-called “home appliance”, and the “home appliance” that is capable of communication with the network 4 may be referred to as “information appliance”.

As already described, the information processing device 3 is capable of receiving (may also be referred to as “acquiring”) the sensor data of the sensor 2 via the network 4 and of processing the sensor data. Thus, the information processing device 3 may also be referred to as sensor data processing device 3.

Based on the received sensor data, the information processing device 3 may assess (may also be referred to as “estimate”) a state such as body movement, heartbeat, or respiration of the user. The information processing device 3 may control the interior environment based on the estimation result as already described.

The sensor 2 is exemplarily capable of sensing living body information of the user in the interior space in a contactless manner. The user in the interior space is one example of a sensing target of the sensor 2. “User” may also be referred to as “observed person” or “subject” by the sensor 2. “Living body information” may be referred to as “vital information”. “Sensing” may be rephrased as “detection” or “measurement”.

One non-limiting example of the vital information is information that indicates the heartbeat, respiration, or movement of body of the user. “Movement of body” of the user may be abbreviated as “body movement” for convenience.

“Body movement” is not limited to movement in activity of the user but may exemplarily include the movement of body in response to the change in the heartbeat or respiration at rest such as sleep of the user. Based on the vital information, for example, it is possible to detect, assess, or estimate the sleep state such as whether the user is asleep or whether the user is awake.

Consequently, the sensor 2 may also be referred to as “contactless type body movement sensor 2” or “contactless type sleep sensor 2” for convenience. An assessment about the sleep state based on the vital information may be abbreviated as “sleep assessment” for convenience. One example of a sleep assessment method will be described later.

“Sensor information” that is transmitted to the information processing device 3 by the sensor 2 may include either one or both of a measurement value, which is a sensing result, and information, which is generated by the sensor 2 based on the measurement value.

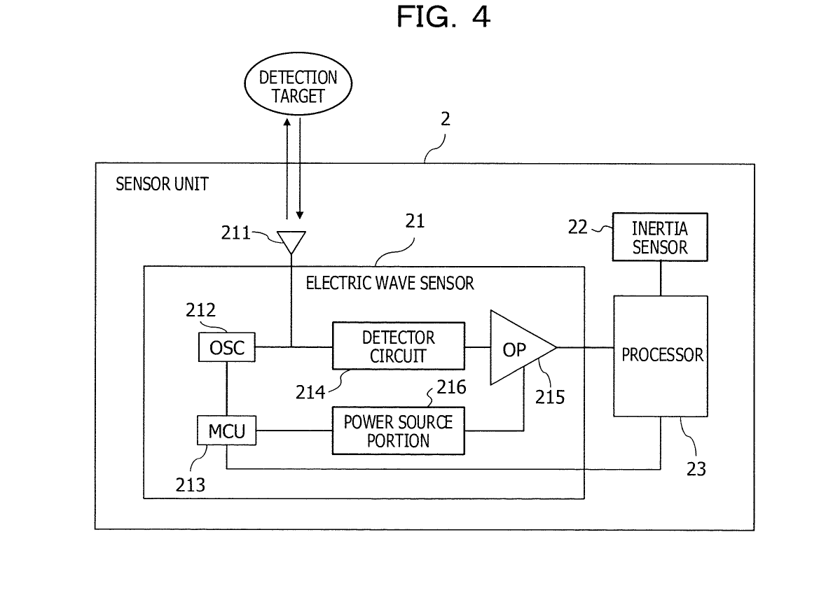

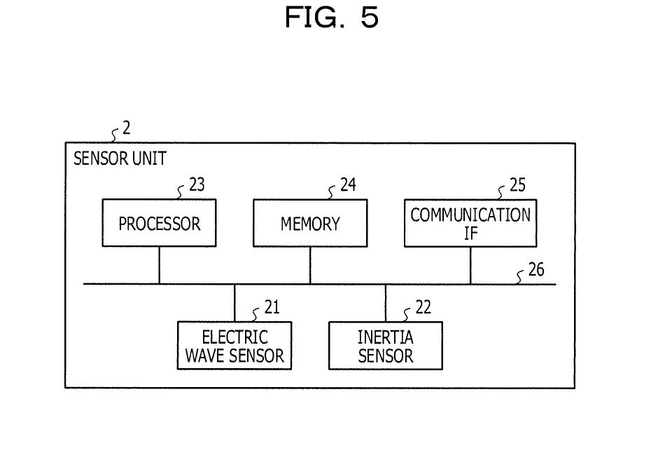

As described later with reference to FIG. 4 and FIG. 5, the sensor 2 may include an electric wave sensor 21 and an inertia sensor 22. The sensor 2 that includes the electric wave sensor 21 and the inertia sensor 22 may also be referred to as “sensor unit 2” for convenience.

The electric wave sensor 21 may irradiate the sensing target with an electric wave such as a microwave and may detect “movement” of the sensing target in a contactless manner based on the change in a reflected wave that is reflected by the sensing target and is received. Note that the electric wave sensor 21 may also be referred to as “Doppler sensor 21”.

For example, in a case where the distance between the electric wave sensor 21 and the sensing target changes, a change occurs to the reflected wave due to the Doppler effect. The change in the reflected wave may exemplarily be considered as the change in either one or both of the amplitude and the frequency of the reflected wave.

In a case where the sensing target is exemplarily a living body such as a human body, the distance between the electric wave sensor 21 and the sensing target changes in response to “movement” of the living body. Thus, the vital information may be sensed.

As already described, “movement” (which may be rephrased as “position change”) of the living body is not limited to physical movement in activity of the living body but may include the movement of the living body surface (for example, skin) in response to the heartbeat or respiration at rest such as sleep of the living body.

The movement of the living body surface may be considered to occur in response to the movement of an organ of the living body. For example, movement occurs to the skin in response to a beat of the heart. Further, movement occurs to the skin in response to expansion and contraction of the lungs that accompany respiration.

Because the change due to the Doppler effect in the reflected wave of the microwave irradiated by the electric wave sensor 21 occurs in response to those kinds of “movement” of the living body, it is possible to sense the vital information that indicates the physical movement, heartbeat, respiration rate, or the like, for example, based on the change.

Based on the vital information that is sensed by the electric wave sensor 21, for example, it is possible to detect, assess, or estimate the sleep state of the living body such as whether the living body is asleep or whether the living body is awake in a contactless manner.

The sensor 2 may exemplarily be arranged in a ceiling or wall of the interior space, in an internal portion or an external portion of the lighting equipment 8 mounted on the ceiling, or in an internal portion or an external portion of the air conditioner 7 mounted on the wall or may be arranged in furniture, bedding (for example, a bed 5), or the like, which is installed in the interior space. As one non-limiting example, FIG. 1 exemplifies that the sensor 2 may be arranged in the ceiling or may be arranged in the bed 5.

A mode in which the sensor 2 is arranged in the ceiling is one example of a mode in which the sensor 2 is arranged in an upper portion of the space in a state where the transmission side of electric wave by the electric wave sensor 21 is directed toward possibility of the space.

Meanwhile, a mode in which the sensor 2 is arranged in the bed 5 is one example of a mode in which the sensor 2 is arranged in a lower portion of the space in a state where the transmission side of electric wave by the electric wave sensor 21 is directed in an upward direction of the space.

Further, a mode in which the sensor 2 is arranged in the wall is one example of a mode in which the sensor 2 is arranged in a lateral portion of the space in a state where the transmission side of electric wave by the electric wave sensor 21 is directed in a lateral direction of the space.

In a mode in which the sensor 2 is arranged in the bed 5, the sensor 2 may be arranged while being associated with the user. For example, in the bed 5, the sensor 2 may be mounted on the bed 5 such that a portion or whole of sleeping region which the user is assumed to occupy during sleep is included in the sensing range.

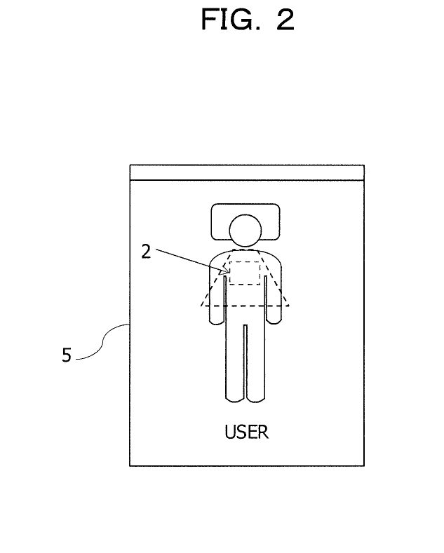

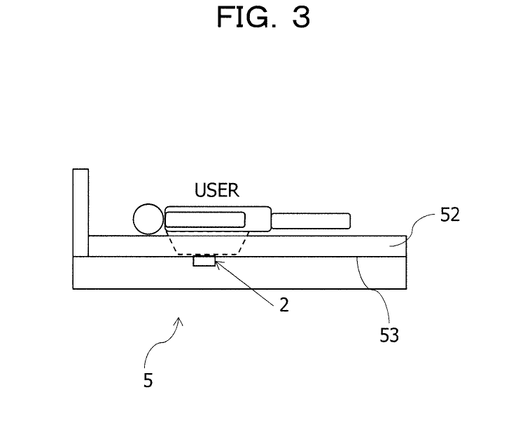

As a non-limiting example, the sensor 2 may be mounted in a position in which the directivity of the transmitted electric wave is formed to the sleeping region of the user and the sensor 2 is capable of irradiating the user with the electric wave. As one example of such a mounting position (which may be referred to as “sensor mounting position” for convenience), as schematically exemplified in FIG. 2 and FIG. 3, a position in which the user may be irradiated with the electric wave from a back side of the bed 5, for example, a back side of a mattress 52 may be raised.

For example, the sensor 2 may be mounted in the region that corresponds to the sleeping region of the user of a floor plate (which may also be referred to as “bottom plate”) 53 (see FIG. 3) of the bed 5 on which the mattress 52 is placed such that the directivity of the transmitted electric wave is directed upward.

The sensing range of the sensor 2 may be set so as to include the chest of the user as schematically exemplified in each of FIG. 2 and FIG. 3. The setting makes it easy to measure the heartbeat or respiration of the user.

As described later, the sensing range of the sensor 2 may be adjusted by controlling the transmission power of the electric wave that is transmitted by the electric wave sensor 21 as described later.

As exemplified in FIG. 2 and FIG. 3, in a mode in which the sensor 2 is mounted on the floor plate 53 of the bed 5, it is easy to make adjustment such that the region that includes at least the chest of the user is included in the sensing range so as to make it easy to measure the heartbeat or respiration of the user.

(Configuration Example of Sensor 2)

Next, a configuration example of the sensor 2 will be described with reference to FIG. 4 and FIG. 5. As illustrated in FIG. 4 and FIG. 5, the sensor 2 may exemplarily include the electric wave sensor 21, the inertia sensor 22, a processor 23, a memory 24, and a communication IF 25.

As exemplified in FIG. 5, the electric wave sensor 21, the inertia sensor 22, the processor 23, the memory 24, and the communication IF 25 may exemplarily be connected together by a communication bus 26 so as to be capable of mutual communication via the processor 23.

The electric wave sensor 21 may be the Doppler sensor 21 and exemplarily generates a beat signal by phase detection of the electric wave, which is transmitted to the space, and the reflected wave of the transmitted electric wave. The beat signal may be given to the processor 23 as an output signal of the electric wave sensor 21.

For example, as illustrated in FIG. 4, the electric wave sensor 21 may include an antenna 211, a local oscillator (OSC) 212, a micro control unit (MCU) 213, a detector circuit 214, an operational amplifier (OP) 215, and a power source portion (or a power source circuit) 216.

The antenna 211 transmits the electric wave with the oscillating frequency generated by the OSC 212 to the space and receives the electric wave as the transmitted electric wave that is reflected by the user positioned in the space (reflected wave). Note that, in the example in FIG. 4, the antenna 211 is shared for transmission and reception but may separately be provided for transmission and reception.

The OSC 212 exemplarily performs an oscillating action in accordance with the control by the MCU 213 and outputs a signal at a prescribed frequency (which may be referred to as “local signal” for convenience). The local signal is transmitted as the transmitted electric wave from the antenna 211 and is input to the detector circuit 214.

The oscillating frequency of the OSC 212 (in other words, the frequency of the electric wave transmitted by the electric wave sensor 21) may exemplarily be a frequency in a microwave band. The microwave band may exemplarily be the 2.4 GHz band or may be the 24 GHz band. Those frequency bands are examples of the frequency bands whose use in indoor places is permitted by the Radio Act of Japan. The frequency bands that conform to the regulations of the Radio Act may be used for the transmitted electric wave of the electric wave sensor 21.

The MCU 213 exemplarily controls the oscillating action of the OSC 212 in accordance with the control by the processor 23.

The detector circuit 214 performs phase detection of the reflected wave that is received by the antenna 211 and the local signal from the OSC 212 (in other words, the transmitted electric wave) and thereby outputs the beat signal. Note that the detector circuit 214 may be substituted by a mixer that mixes the transmitted electric wave and the reflected wave. Mixing by the mixer may be considered as equivalent to phase detection.

Here, the beat signal obtained by the detector circuit 214 exhibits an amplitude change and a frequency change due to the Doppler effect in response to the physical change such as the heartbeat, respiration, or body movement of the user.

For example, as the physical change amount (in other words, the relative velocity to the Doppler sensor 21) of the user in the interior space becomes larger, the frequency and amplitude value of the beat signal tend to become higher. In other words, the beat signal includes information that indicates the physical change such as the heartbeat, respiration, or body movement of the user.

The operational amplifier 215 amplifies the beat signal that is output from the detector circuit 214. The amplified beat signal is input to the processor 23.

The power source portion 216 exemplarily supplies driving power to the MCU 213, the detector circuit 214, and the operational amplifier 215.

Meanwhile, the inertia sensor 22 may exemplarily detect the orientation (which may also be referred to as “mounting angle” or “installation angle”) of the sensor 2 with respect to the gravity direction as a reference.

The inertia sensor 22 may be an acceleration sensor or may be a gyroscope. Exemplarily, a sensor of either one of a piezoelectric type and a capacitive type may be applied to the acceleration sensor. A sensor of any of a rotation mechanical (top) type, an optical type, and a vibration type may be applied to the gyroscope.

The inertia sensor 22 may have one or plural detection axes. A gravity component along the detection axis may be detected as “acceleration”, for example.

At least one of the detection axes of the inertia sensor 22 may be directed in the direction of the directivity of the electric wave transmitted from the electric wave sensor 21 to the outside of the sensor unit 2 (which may be referred to as “electric wave irradiation direction” for convenience).

In other words, at least one of the detection axes of the inertia sensor 22 may be directed in the direction along the direction from the electric wave sensor 21 as an electric wave transmission source toward an electric wave irradiation surface or an electric wave irradiation side of the sensor unit 2.

A detection signal of the inertia sensor 22 may be input to the processor 23.

The processor 23 is one example of an arithmetic processor that includes an arithmetic processing capability. The arithmetic processor may be referred to as arithmetic processing device or arithmetic processing circuit. Exemplarily, an integrated circuit (IC) such as a micro processing unit (MPU) or a digital signal processor (DSP) may be applied to the processor 23 as one example of the arithmetic processor. Note that “processor” may also be referred to as “control portion” or “computer”.

The processor 23 may detect the vital information of the user in the interior space based on the detection signal of the electric wave sensor 21 and may assess the sleep state of the user based on the vital information.

The detection signal of the inertia sensor 22 may be used for transmission power control of the electric wave that is transmitted by the electric wave sensor 21. Further, the detection signal of the inertia sensor 22 may be used for correction of the detection signal of the electric wave sensor 21 or information obtained based on the detection signal (which may generically be referred to as “correction of sensor information” for convenience) or correction of a threshold value that is used for body movement detection or the sleep assessment. Specific examples of correction processes will be described later.

Note that both of the detection signal of the electric wave sensor 21 and the detection signal of the inertia sensor 22 may also be referred to as “detection value” or “output value”. Further, the detection value of the electric wave sensor 21 may be referred to as “electric wave sensor value” for convenience, and the detection value of the inertia sensor 22 may be referred to as “inertia sensor value” for convenience.

Further, a portion or all of the transmission power control of the electric wave sensor 21, the detection of the vital information, the sleep state assessment of the user, the correction of the sensor information, and the correction of the threshold value, which are described above, may exemplarily be conducted in the sensor unit 2 or may be conducted in the information processing device 3.

Next, in FIG. 5, the memory 24 is one example of a storage medium and may be a random access memory (RAM), a flash memory, or the like. In the memory 24, a program or data that are read and used by the processor 23 for an action may be stored. “Program” may be referred to as “software” or “application”. “Data” may include the data that are generated in accordance with the action of the processor 23.

The communication IF 25 is one example of a communication portion included in the sensor unit 2, is exemplarily connected with the router 6 (see FIG. 1), and enables communication with the information processing device 3 via the network 4.

For example, the communication IF 25 may transmit either one or both of the detection signals of the electric wave sensor 21 and the inertia sensor 22 or information that is obtained based on either one or both of the detection signals to the information processing device 3.

In other words, the sensor information transmitted from the sensor 2 to the information processing device 3 may include either one or both of the measurement values of the electric wave sensor 21 and the inertia sensor 22 or may include information that is obtained based on either one or both of the measurement values.

Note that the sensor unit 2 may receive power supply from the outside. For example, the sensor unit 2 may receive power supply from an alternate current (AC) power source that is included in the interior space or may receive power supply from a power source that is included in the air conditioner 7, the lighting equipment 8, or the bed 5. In other words, the power source for the sensor unit 2 may be shared as the power source for the air conditioner 7, the lighting equipment 8, or the bed 5.

However, in a case where power is fed to the sensor unit 2 from a separate power source from the power source for the air conditioner 7, the lighting equipment 8, or the bed 5, even if the power source for the air conditioner 7, the lighting equipment 8, or the bed 5 is turned off, the sensor unit 2 is capable of sensing.

In other words, the sensor unit 2 may be operable as the sensor unit 2 alone even in a state where power for the air conditioner 7, the lighting equipment 8, or the bed 5 is not supplied and may thus be used as “monitoring function”.

Note that as one non-limiting example, a Universal Serial Bus (USB) may be applied to the connection between the sensor unit 2 and the power source for the air conditioner 7, the lighting equipment 8, or the bed 5. For example, in a case where a USB port that is capable of power supply is included in the air conditioner 7, the lighting equipment 8, or the bed 5, the sensor unit 2 may detachably be connected with the USB port by a USB cable.

(Configuration Example of Information Processing Device 3)

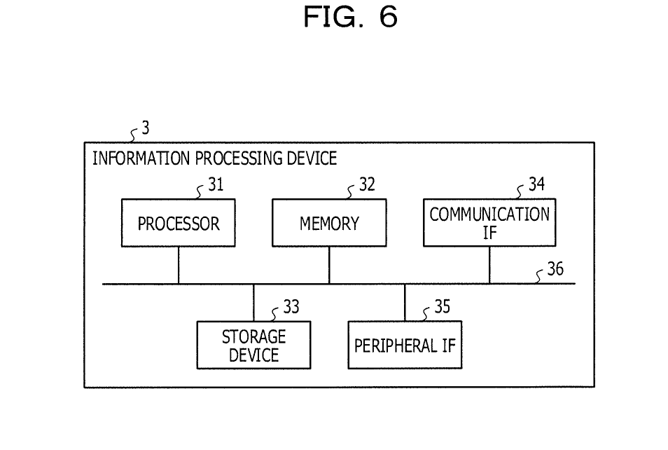

Next, a configuration example of the information processing device 3 exemplified in FIG. 1 will be described with reference to FIG. 6. As illustrated in FIG. 6, the information processing device 3 may exemplarily include a processor 31, a memory 32, a storage device 33, a communication interface (IF) 34, and a peripheral IF 35.

The processor 31, the memory 32, the storage device 33, the communication IF 34, and the peripheral IF 35 may exemplarily be connected together by a communication bus 36 so as to be capable of mutual communication via the processor 31.

The processor 31 is one example of an arithmetic processor that includes an arithmetic processing capability. The arithmetic processor may be referred to as arithmetic processing device or arithmetic processing circuit. Exemplarily, an IC such as a CPU or an MPU or a DSP may be applied to the processor 31 as one example of the arithmetic processor.

Exemplarily, the processor 31 is one example of a control portion (or a computer) that controls a general action of the information processing device 3. Control by the processor 31 may include controlling the communication via the network 4. By the control of the communication, remote control of the air conditioner 7 or the lighting equipment 8, for example, may be performed via the network 4.

The processor 31 may exemplarily conduct a portion or all of the already-described processes, which may be conducted by the processor 23 of the sensor 2, based on the sensor information of the sensor 2 that is received by the communication IF 34. Examples of the already-described processes are the transmission power control of the electric wave sensor 21, the correction of the sensor information, the correction of the threshold value used for the body movement detection or the sleep assessment, and so forth.

Further, the processor 31 may exemplarily generate a control signal for the sensor 2. For example, the processor 31 may generate a control signal that controls the transmission power of the electric wave transmitted by the electric wave sensor 21 based on the detection information of the inertia sensor 22 that is acquired from the sensor 2.

In addition, the processor 31 may exemplarily generate a control signal for controlling the environment of the space in which the sensor 2 is installed, for example, a control signal for controlling the action of the air conditioner 7 or the lighting equipment 8. The control signal may exemplarily be generated based on the sensor information acquired from the sensor 2 or a state assessment result about sleep of the user based on the sensor information.

The control signal generated by the processor 31 may exemplarily be transmitted to the sensor 2, the air conditioner 7, the lighting equipment 8, or the like via the communication IF 34.

The memory 32 is one example of a storage medium and may be a RAM, a flash memory, or the like. In the memory 32, a program or data that are read and used by the processor 31 for an action may be stored.

The storage device 33 may store various kinds of data or programs. For the storage device 33, a hard disk drive (HDD), a solid state drive (SSD), a flash memory, or the like may be used.

Data that are stored in the storage device 33 may exemplarily include the sensor information of the sensor 2, the vital information obtained based on the sensor information, an assessment result about the sleep state estimated based on the vital information, and so forth, which are received by the communication IF 34.

The data stored in the storage device 33 may properly be formed into a database (DB). The data formed into the DB may be referred to as “cloud data”, “big data”, or the like. Note that the storage device 33 and the memory 32 may also generically referred to as “storage portion”.

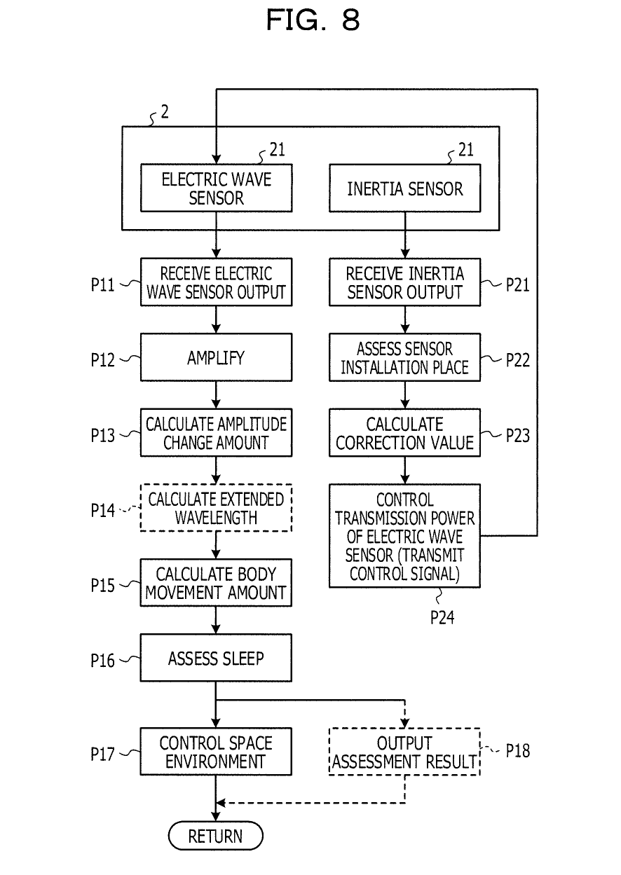

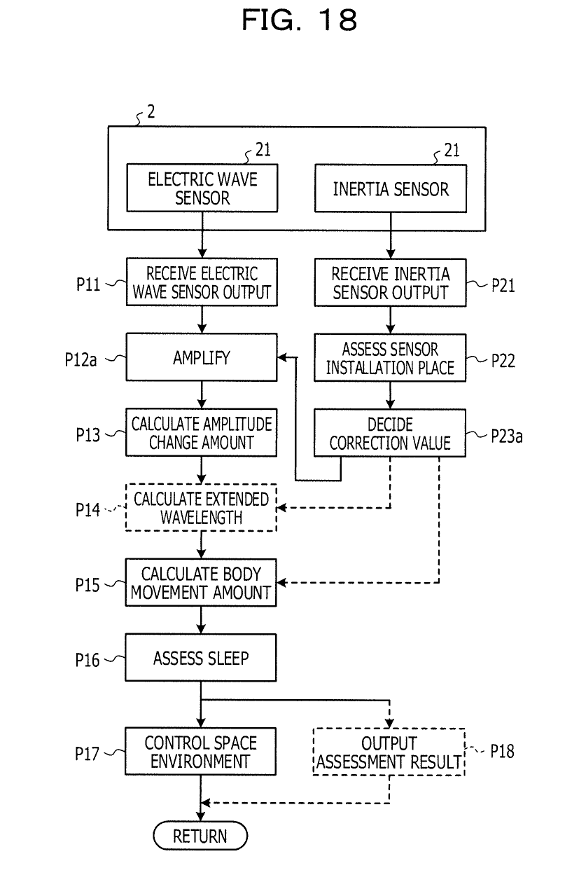

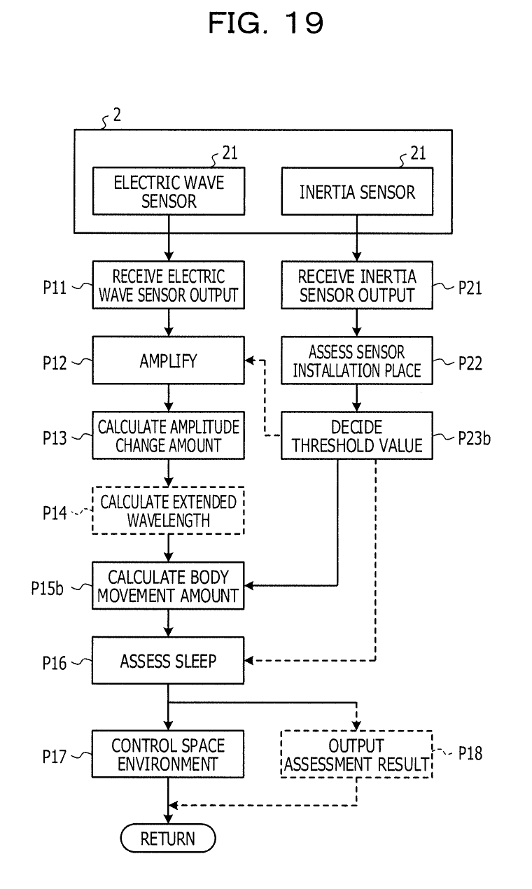

Programs stored in the storage device 33 may include programs that execute processes which will be described later with reference to FIG. 8, FIG. 18, and FIG. 19.

The program that executes the process described later with reference to FIG. 8 may be referred to as “sensor control program” for convenience. The programs that execute the processes described later with reference to FIG. 18 and FIG. 19 may be referred to as “sensor data processing program” for convenience.

A portion or all of program codes that form the program may be stored in the storage portion or may be described as a portion of an operating system (OS).

The programs or data may be provided in a form in which those are recorded in a computer-readable recording medium. As examples of the recording medium, a flexible disk, a CD-ROM, a CD-R, a CD-RW, an MO, a DVD, a Blu-ray disk, a portable hard disk, and so forth may be raised. Further, a semiconductor memory such as a Universal Serial Bus (USB) memory is one example of the recording medium.

Instead, the programs or data may be provided (downloaded) from a server or the like to the information processing device 3 via the network 4. For example, the programs or data may be provided for the information processing device 3 through the communication IF 34. Further, the programs or data may be input to the information processing device 3 from an input apparatus or the like that is connected with the peripheral IF 35 and will be described later.

The communication IF 34 is one example of a communication portion included in the information processing device 3, is exemplarily connected with the network 4, and enables communication via the network 4.

Focusing on a reception process, the communication IF 34 is one example of a reception portion (which may also be referred to as “acquisition portion”) that receives the information which is transmitted by the sensor 2 to the information processing device 3.

Meanwhile, focusing on a transmission process, the communication IF 34 is one example of a transmission portion that transmits the control signal generated by the processor 31, for example, to the sensor 2, the air conditioner 7, or the lighting equipment 8. Exemplarily, an Ethernet® card may be applied to the communication IF 34.

The peripheral IF 35 is exemplarily an interface for connecting a peripheral apparatus with the information processing device 3.

The peripheral apparatus may include an input apparatus for inputting information to the information processing device 3 and an output apparatus for outputting information generated by the information processing device 3.

The input apparatus may include a keyboard, a mouse, a touch panel, and so forth. The output apparatus may include a display, a printer, and so forth.

Incidentally, in a case where the sensor 2 that has the electric wave sensor 21 is arranged in the interior space, the average distance between the sensor 2 and the user may be different depending on the arrangement position. For example, between a case where the sensor 2 is installed in the ceiling and a case where the sensor 2 is installed in the bed 5, the average distance between the sensor 2 and the user may be longer in a case of ceiling installation than a case of bed installation.

The electric wave transmitted by the electric wave sensor 21 is attenuated as the propagation distance becomes longer. Thus, in a case where the process for the transmission power or a received wave of the electric wave sensor 21 is not appropriately selected in accordance with the distance between the sensor 2 and the user, the body movement detection precision of the user and further the sleep assessment precision of the user may lower.

For example, in a case where the transmission power of the electric wave sensor 21 is too weak with respect to the distance between the sensor 2 and the user, it is possible that the detection signal level of the electric wave sensor 21 does not reach a level that is sufficient for detection of the body movement of the user.

Conversely, in a case where the transmission power of the electric wave sensor 21 is too strong with respect to the distance between the sensor 2 and the user, in the received wave, it becomes easy for a component corresponding to movement of an object or a person, which is different from a body movement component of the user as the sensing target, to enter the detection signal of the electric wave sensor 21. Instead, the reception power of the reflected wave becomes too high, the detection signal of the electric wave sensor 21 is saturated, and it thereby becomes possible that the body movement component of the user may not be detected.

Consequently, the transmission power or the detection signal level of the electric wave sensor 21 may be considered as one example of a parameter that decides the detection sensitivity of the body movement component of the user. In a case where excess or deficiency in the transmission power or the detection signal level of the electric wave sensor 21 is present with respect to the distance between the sensor 2 and the user, excess or deficiency may occur to the detection sensitivity of the body movement component. As a result, the body movement detection precision of the user and further the sleep assessment precision of the user may lower.

Further, for example, in a case where the body movement detection or the sleep assessment is performed by a comparison between the detection signal of the electric wave sensor 21 (or information obtained based on the detection signal) and a threshold value, the precision of the body movement detection or the sleep assessment may lower in a case where the threshold value corresponding to the assumed distance between the sensor 2 and the user is not set.

For example, it is possible that the body movement detection or the sleep assessment becomes difficult or that a noise component other than the body movement component is falsely detected as if the noise component were the body movement component. Further, it is possible that the user in activity is falsely assessed as sleeping or that the sleeping user is falsely assessed as being in activity.

Consequently, the threshold value used for the body movement detection or the sleep assessment may be considered as another example of the parameter that decides the detection sensitivity of the body movement component of the user. In a case where the threshold value corresponding to the distance between the sensor 2 and the user is not appropriately set, excess or deficiency may occur to the sensitivity of the body movement component or sleep assessment. As a result, the body movement detection precision of the user and further the sleep assessment precision of the user may lower.

In a case where the sensors 2 in which the transmission power or the detection signal level of the electric wave sensor 21 is adjusted are prepared in advance for different assumed distances between the sensors 2 and the user, for example, in different installation places of the sensors 2 and the installed sensors 2 are thereby used in different manners, occurrence of excess or deficiency in the detection sensitivity might be avoided.

For example, the sensor 2 that is adjusted for the ceiling installation and the sensor 2 that is adjusted for the bed installation are prepared, the sensor 2 for the ceiling installation is installed in the ceiling, and the sensor 2 for the bed installation is installed in the bed 5. However, preparing the sensors 2 for different installation places is disadvantageous in view of manufacturing cost, inventory cost, or the like.

In a case where the transmission power or the detection signal level of the electric wave sensor 21 is made adjustable in installation of the sensor 2, for example, one sensor 2 may be shared by plural installation places. However, adjustment work occurs in the installation and is thus troublesome for an installer. Further, there may be a case where a switch or the like for switching the transmission power or the detection signal level in accordance with the installation place of the sensor 2 has to be provided to the sensor 2.

In addition, as for the sensors 2 for different installation places or the sensor 2 for any installation place, trouble for registering information or the like by which the installation place of the sensor 2 is identifiable may occur to a device that processes or analyzes the sensor information (which may be either one of the sensor 2 or the information processing device 3, for example).

For example, the information or the like is for enabling the threshold value of the detection signal level or the body movement detection of the electric wave sensor 21 to be corrected in accordance with the installation place of the sensor 2, in other words, in accordance with the assumed distance between the sensor 2 and the user.

In other words, trouble for altering an analysis process or an analysis algorithm of the sensor information in accordance with the installation place of the sensor 2 may occur. Further, a situation is possible in which what kind of place the sensor 2 is installed in may not be grasped in advance and the information by which the installation place of the sensor 2 is identifiable may not be given to the sensor 2 or the information processing device 3 in advance.

Because of the above trouble or situation and in relation to the installation place of the sensor 2, constraint on the degree of freedom or flexibility may occur about settings about the sensor 2 or the information processing device 3, a change in the layout of the interior space, or the like, for example.

Accordingly, in this embodiment, the installation place of the sensor 2 is detected, assessed, or estimated by using the inertia sensor 22 included in the sensor 2. In accordance with the result, the detection sensitivity of the electric wave sensor 21 is controlled, and an improvement in the body movement detection precision of the user and further the sleep assessment precision is thereby intended.

The installation place of the sensor 2 may exemplarily be detected, assessed, or estimated based on the orientation of the sensor 2 with respect to the gravity direction, which is detected by the inertia sensor 22, as a reference.

For example, as already described, cases where at least one detection axis of the inertia sensor 22 is directed in the direction along the electric wave irradiation direction of the electric wave sensor 21 and where the sensor 2 is installed in the ceiling, where the sensor 2 is installed in the bed 5, and where the sensor 2 is installed in the wall are assumed.

Note that the installation in the ceiling is one example of the mode in which the sensor 2 is installed in an upper portion of the interior space as the sensing target of the sensor 2, and the installation in the bed 5 is one example of the mode in which the sensor 2 is installed in a lower portion of the interior space. The installation in the wall is one example of the mode in which the sensor 2 is installed in a lateral portion of the interior space.

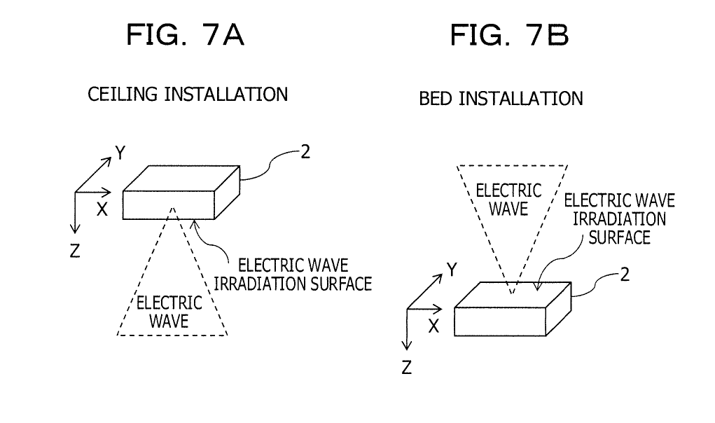

In a case where the sensor 2 is installed in the ceiling, as schematically exemplified in FIG. 7A, the electric wave irradiation surface of the electric wave sensor 21 of the sensor 2 is directed in a downward direction of the interior space (for example, the positive direction of a Z axis). Thus, the gravity direction detected by the inertia sensor 22 (for example, the positive direction of the Z axis) is directed toward the same side as the side to which the electric wave is transmitted from the sensor 2 by the electric wave sensor 21.

Meanwhile, in a case where the sensor 2 is installed in the back side of the bed 5 as exemplified in FIG. 2 and FIG. 3, for example, as schematically exemplified in FIG. 7B, the electric wave irradiation surface of the electric wave sensor 21 of the sensor 2 is directed in an upward direction of the interior space (the negative direction of the Z axis). Thus, the gravity direction detected by the inertia sensor 22 (the positive direction of the Z axis) is directed toward the opposite side to the side to which the electric wave is transmitted from the sensor 2 by the electric wave sensor 21.

In such a manner, between a case where the sensor 2 is installed in the upper portion of the space such as the ceiling and a case where the sensor 2 is installed in the lower portion of the space such as the bed 5, the gravity direction detected by the inertia sensor 22 included in the sensor 2 is inverted.

Further, in a case where the sensor 2 is installed in the lateral portion such as the wall of the interior space, the gravity direction detected by the inertia sensor 22 is directed in a direction that is offset from or a direction that is orthogonal to the direction in which the electric wave is transmitted from the electric wave sensor 21 in the sensor 2.

Consequently, based on the gravity direction that is detected by the inertia sensor 22, the difference among whether the sensor 2 is installed in the upper portion (for example, the ceiling) of the space, whether the sensor 2 is installed in the lower portion (for example, the bed 5) of the space, and whether the sensor 2 is installed in the lateral portion (for example, the wall) of the space may be assessed or identified.

Control of the detection sensitivity of the electric wave sensor 21 based on the above assessment result may include controlling or correcting the parameter that causes a change in the detection sensitivity as already described.

For example, at least any of the transmission power of the electric wave sensor 21, the detection signal level of the electric wave sensor 21, and the threshold value used for the body movement detection or the sleep assessment is controlled or corrected, and the detection sensitivity of the electric wave sensor 21 may thereby be controlled.

Note that both of the correction of the detection signal level of the electric wave sensor 21 and the correction of the threshold value may be considered as examples of changing a process about the reflected wave of the transmitted electric wave by the electric wave sensor 21.

In other words, the information processing device 3 may change the process about the reflected wave of the transmitted electric wave by the electric wave sensor 21 in accordance with the gravity direction detected by the inertia sensor 22.

### ACTION EXAMPLES

In the following, several action examples of the sensor system 1 according to one embodiment will be described.

For example, an action example of controlling the transmission power of the electric wave sensor 21, an action example of correcting the detection signal level of the electric wave sensor 21, and an action example of correcting the threshold value in accordance with the gravity direction detected by the inertia sensor 22 will respectively be described as first to third examples.

### First Example

FIG. 8 is a flowchart that illustrates an action example of the sensor system 1 according to the first example. The flowchart exemplified in FIG. 8 may exemplarily be executed by the information processing device 3 (for example, the processor 31).

The information processing device 3 receives a sensor value of the electric wave sensor 21 from the sensor 2 (process P11). Further, the information processing device 3 receives the inertia sensor value from the inertia sensor 22 (process P21).

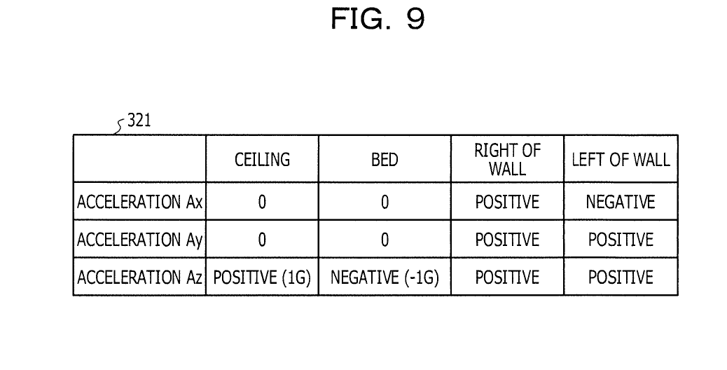

The information processing device 3 may assess the installation angle of the sensor 2 based on the inertia sensor value (process P22). For the assessment, an installation place categorization table 321 illustrated in FIG. 9 may exemplarily be used. The installation place categorization table 321 may exemplarily be stored in the memory 32 of the information processing device 3.

The installation place categorization table 321 is one example of information in which the installation places of the sensor 2 are categorized by the combinations of the detection values of the respective detection axes of the inertia sensor 22. For example, the installation place categorization table 321 indicates which of the upper portion (for example, the ceiling), the lower portion (for example, the bed 5), and the lateral portion (for example, the wall) in the interior space the sensor 2 is installed in.

As one non-limiting example, the inertia sensor 22 is a three-axis sensor that has three detection axes of X, Y, and Z, which are mutually orthogonal, and accelerations Ax, Ay, and Az in the directions along the individual detection axes X, Y, and Z are respectively detected as the inertia sensor values.

As schematically exemplified in FIG. 1, in a case where the interior space is expressed as a cuboid for convenience and the perpendicularly downward direction is associated with the positive direction of the Z axis, the ceiling and the floor may be considered as planes that are parallel with an XY plane, and the wall may be considered as a plane that is parallel with an XZ plane or a YZ plane.

In a case where the sensor 2 is installed in the ceiling such that the electric wave irradiation surface is directed in the perpendicularly downward direction, the inertia sensor values (Ax, Ay, Az) indicate (Ax, Ay, Az)=(0, 0, +1G). Note that “G” denotes the gravitational acceleration.

In this case, the gravity direction detected based on the inertia sensor values is on the same side as the side from which the electric wave is transmitted from the sensor 2. Thus, the processor 31 as one example of the control portion in the information processing device 3 may assess that the sensor 2 is installed in the ceiling.

On the other hand, in a case where the sensor 2 is installed in the bed 5 such that the electric wave irradiation surface is directed in the perpendicularly upward direction, the inertia sensor values (Ax, Ay, Az) indicate (Ax, Ay, Az)=(0, 0, −1G). In this case, the gravity direction detected based on the inertia sensor values is on the opposite side to the side from which the electric wave is transmitted from the sensor 2. Thus, the processor 31 may assess that the sensor 2 is installed in the bed 5.

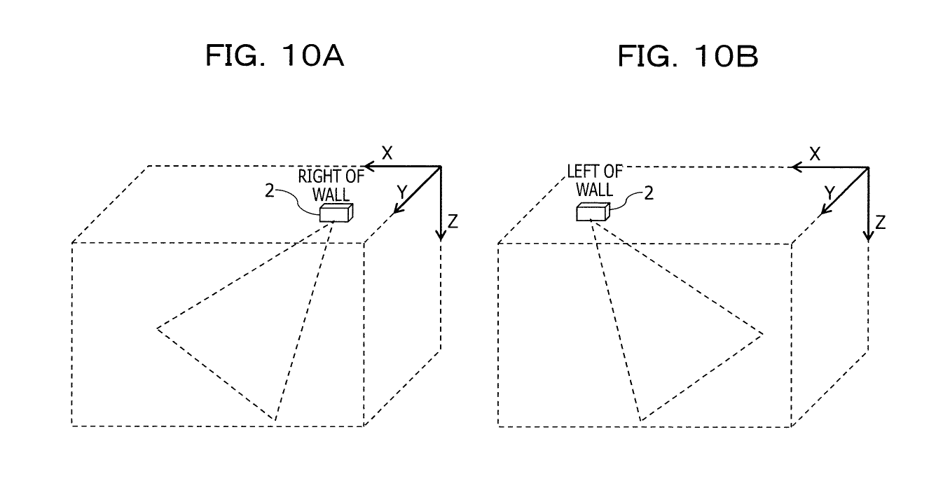

Further, as schematically exemplified in FIG. 10A, in a case where the sensor 2 is installed in the wall such that the electric wave irradiation surface of the sensor 2 is directed in an obliquely downward direction from an upper right side of the wall toward the center of the interior space, all of the inertia sensor values Ax, Ay, and Az take positive values. That is, (Ax, Ay, Az)=(positive, positive, positive) is provided.

Meanwhile, as schematically exemplified in FIG. 10B, in a case where the sensor 2 is installed in the wall such that the electric wave irradiation surface of the sensor 2 is directed in an obliquely downward direction from an upper left side of the wall toward the center of the interior space, the inertia sensor value Ax takes a negative value, and both of the inertia sensor values Ay and Az become positive values. That is, (Ax, Ay, Az)=(negative, positive, positive) is provided.

In the cases of FIG. 10A and FIG. 10B, the gravity direction detected based on the inertia sensor values is neither on the same side as nor on the opposite side to the side from which the electric wave is transmitted from the sensor 2. Thus, the processor 31 may assess that the sensor 2 is installed in the wall.

In such a manner, the information processing device 3 refers to the installation place categorization table 321 based on the inertia sensor values (Ax, Ay, Az) and may thereby easily assess or identify which of the ceiling, the bed 5, and the wall (right side and left side) in the interior space the sensor 2 is installed in, with a low load.

Based on the assessment result, the information processing device 3 may control the electric wave sensor 21 such that the transmission power of the electric wave transmitted by the electric wave sensor 21 becomes appropriate power (detection sensitivity, in other words) corresponding to the installation place of the sensor 2 (processes P23 and P24 in FIG. 8).

For example, in the interior space, the lateral distance (for example, the distance between the opposed walls) is often longer than the distance in the perpendicular direction (in other words, the distance between the ceiling and the floor). Thus, when the transmission power of the electric wave sensor 21 in a case where the sensor 2 is installed in the lateral portion of the interior space is set as reference power, the transmission power of the electric wave sensor 21 in a case where the sensor 2 is installed in the ceiling may be lower than the reference power.

Further, in a case where the sensor 2 is installed in the bed 5, the distance between the user as the sensing target and the sensor 2 is often short compared to a case where the sensor 2 is installed in the ceiling. Thus, the transmission power of the electric wave sensor 21 in a case where the sensor 2 is installed in the bed 5 may be lower than the transmission power in a case where the sensor 2 is installed in the ceiling.

Consequently, the processor 31 may perform control for making the transmission power of the electric wave sensor 21 higher than a case where the sensor 2 is installed in the bed 5, in response to the assessment that the sensor 2 is installed in the ceiling that is positioned in the upper portion of the interior space. By the control, a circumstance in which the body movement amount of the user is detected as excessively small or the detection becomes difficult may be inhibited or avoided.

Conversely, the processor 31 may perform control for making the transmission power of the electric wave sensor 21 lower than a case where the sensor 2 is installed in the ceiling, in response to the assessment that the sensor 2 is installed in the bed 5 that is positioned in the lower portion of the interior space. By the control, a circumstance in which the body movement amount of the user is detected as excessively large or the detection becomes difficult due to saturation of the detection signal of the electric wave sensor 21 may be inhibited or avoided.

Further, the processor 31 may perform control for making the transmission power of the electric wave sensor 21 higher than a case where the sensor 2 is installed in the bed 5 or the ceiling, in response to the assessment that the sensor 2 is installed in the wall that is positioned in the lateral portion of the interior space. By the control, a circumstance in which the body movement amount of the user is detected as excessively small or the detection becomes difficult may be inhibited or avoided.

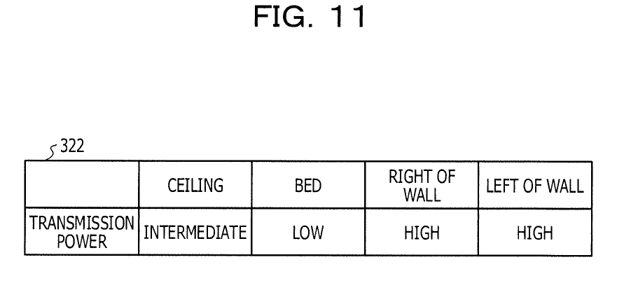

For convenience, in a case where the reference power in wall installation is expressed as “high”, the transmission power in the ceiling installation is expressed as “intermediate”, and the transmission power in the bed installation is expressed as “low”, the transmission power of the electric wave sensor 21 that corresponds to the installation place of the sensor 2 may be represented by a transmission power control table 322 of FIG. 11.

Exemplarily, based on the transmission power control table 322, the information processing device 3 may calculate the correction value of the transmission power of the electric wave sensor 21 that corresponds to the installation place of the sensor 2 (process P23 in FIG. 8). Then, the information processing device 3 may generate a transmission power control signal of the electric wave sensor 21 that corresponds to the calculated correction value and transmit the transmission power control signal to the sensor 2 (process P24).

The transmission power control signal is received by the processor 23 (see FIG. 6) of the sensor 2, for example, and the processor 23 controls the MCU 213 of the electric wave sensor 21 in accordance with the transmission power control signal and thereby controls the power of the transmitted electric wave by the OSC 212.

Accordingly, the transmission power of the electric wave transmitted from the electric wave sensor 21 is controlled to the appropriate power for the installation place of the sensor 2, and the detection sensitivity of the electric wave sensor 21 is controlled to the appropriate sensitivity for the installation place of the sensor 2.

Consequently, a circumstance in which the body movement amount of the user is detected as excessively small or excessively large or the detection becomes difficult due to farness or closeness of the distance of the user with respect to the sensor 2 may be avoided or inhibited.

Note that the above-described examples are examples where the information processing device 3 acquires the measurement values of the inertia sensor 22, as examples of the detection information of the inertia sensor 22, from the sensor 2. Alternatively, the information processing device 3 may acquire information obtained based on the measurement values of the inertia sensor 22, for example, information that indicates the gravity direction or information that indicates the difference in the installation place of the sensor 2, from the sensor 2.

In other words, the sensor 2 may transmit the measurement values themselves of the inertia sensor 22 to the information processing device 3 or may transmit information obtained based on the measurement values, the information enabling the information processing device 3 to specify the installation place of the sensor 2, to the information processing device 3.

As described above, the information processing device 3 is capable of the transmission power control of the electric wave sensor 21 that corresponds to the installation place of the sensor 2 based on the information which is acquired from the sensor 2 and enables the installation place of the sensor 2 to be specified.

In a state where the detection sensitivity of the electric wave sensor 21 is optimized in accordance with the installation place of the sensor 2 by the transmission power control of the electric wave sensor 21, the information processing device 3 may perform body movement amount detection or the sleep state assessment of the user based on the electric wave sensor value that is received from the sensor 2.

For example, the information processing device 3 may properly amplify the electric wave sensor value that is received from the sensor 2 (process P12), calculate the amplitude change amount of the electric wave sensor value (process P13), and calculate “extended wavelength” based on the amplitude change amount (process P14). Further, the information processing device 3 may calculate “body movement amount” of the user based on “extended wavelength” (process P15).

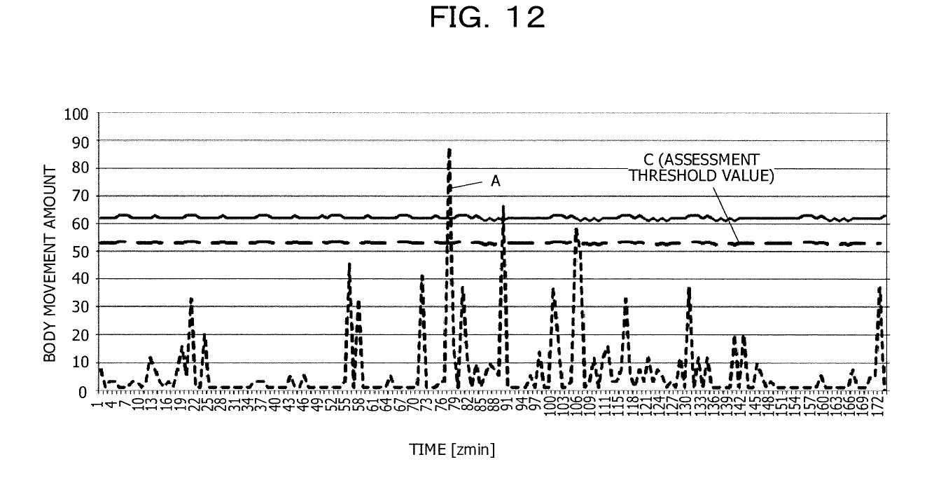

FIG. 12 is a diagram that illustrates one example of the time change in the body movement amount as one example of the vital information which is obtained based on the electric wave sensor value. In FIG. 12, the signal waveform that is indicated by dotted lines A illustrates one example of the time change in the body movement amount, and dotted line C indicates the threshold value of the body movement amount at which an assessment of “body movement present” or “awake” is made (which may be referred to as “assessment threshold value”).

Exemplarily, the user may be assessed as awake in a case where the body movement amount exceeds the assessment threshold value, and the user may be assessed as asleep in a case where the body movement amount is less than the assessment threshold value.

The body movement amount may be considered as a time change in the electric wave sensor value. For example, in a case where the user as the sensing target is awake and in activity, the body movement of the sensing target appears as changes in the amplitude value and the frequency in the electric wave sensor value. For example, as the body movement amount of the user becomes larger, the amplitude value and the frequency of the electric wave sensor value tend to become higher.

In a case where the user is at rest such as sleeping, the change in the heartbeat or respiration becomes dominant in the body movement of the user. Thus, the amplitude value of the electric wave sensor value may be considered as not changing or as almost ignorable in a case where a change is present.

Consequently, the body movement due to the change in the heartbeat or respiration may be considered to appear as a frequency change of the electric wave sensor value. For example, as heart rate or respiration rate increases, the frequency of the electric wave sensor value tends to become higher.

Thus, the body movement amount may be detected based on the changes in the amplitude value and the frequency of the electric wave sensor value. The changes in the amplitude value and the frequency of the electric wave sensor value may be considered as the change in the length of the signal waveform illustrated in FIG. 12 (see dotted lines A) that is extended as a straight line in the time region.

The length of the signal waveform that is extended as a straight line in the time region may be referred to as “extended wavelength” for convenience. Consequently, “extended wavelength” is a different concept from usual “wavelength”. “Extended wavelength” may also be considered to correspond to the length of the locus that is drawn by the electric wave sensor value in a certain unit time in the time region. Note that the unit time may be “second” as a unit or may be “minute” as a unit.

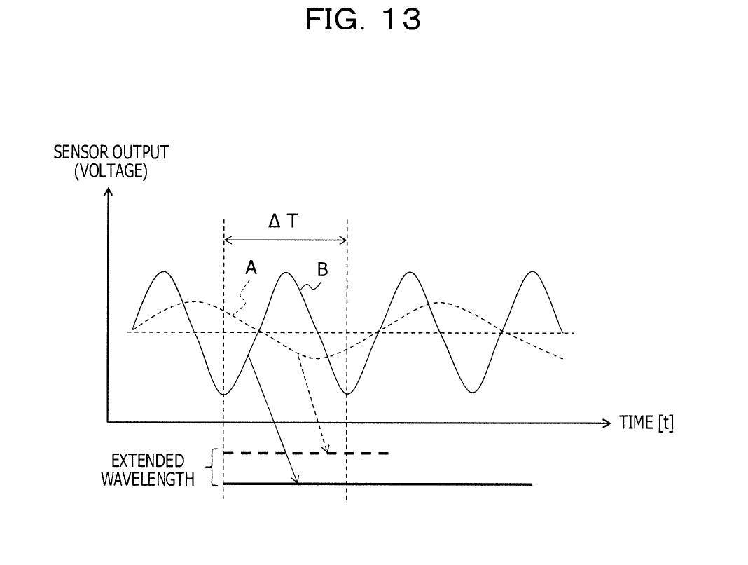

FIG. 13 schematically exemplifies the concept of “extended wavelength”. The horizontal axis of FIG. 13 represents time (t), and the vertical axis of FIG. 13 represents the electric wave sensor value (for example, a voltage [V]).

In FIG. 13, exemplarily, the signal waveform indicated by dotted line A schematically indicates the time change of the electric wave sensor value in a case where the user as the sensing target is sleeping. In FIG. 13, the signal waveform indicated by solid line B schematically indicates the time change of the electric wave sensor value in a case where the user as the sensing target is awake and in activity.

As exemplified in a lower portion of FIG. 13, “extended wavelength” corresponds to the length of the signal waveform per unit time (ΔT) indicated by dotted line A and solid line B, which is extended as a straight line in the time direction.

Exemplarily, “extended wavelength” may be calculated by successively storing the electric wave sensor value in certain periods (which may be referred to as “sampling period”) in the memory 32 (see FIG. 6) and by adding the change amounts of the amplitude value through the unit time.

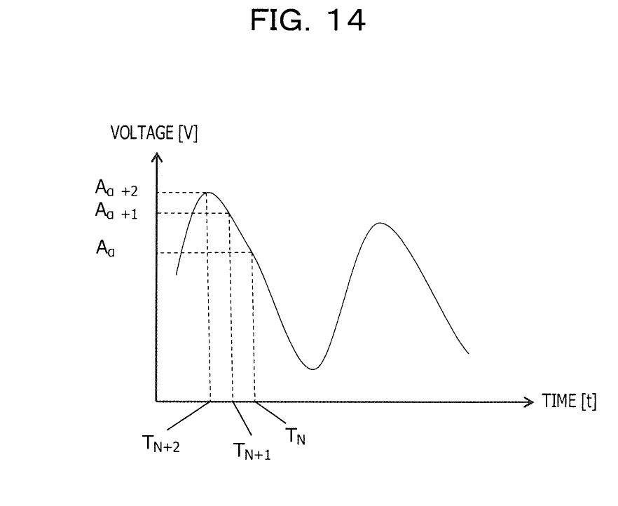

A calculation example of “extended wavelength” will be described with reference to FIG. 14. The horizontal axis of FIG. 14 represents time (t), and the vertical axis of FIG. 14 represents the electric wave sensor value (for example, the voltage [V] that corresponds to the amplitude value).

In the signal waveform exemplified in FIG. 14, at certain timings t=T_{N+2}, t=T_{N+1}, and t=T_{N}, the Doppler sensor values are respectively “A_{α+2}”, “A_{α+1}”, and “A_{α}”.

Note that “N” is an integer that denotes the label of the timing. “A” is a real number that may be taken by a voltage value [V], and “α” is an integer that denotes the label of the voltage value. Each of the timings t=T_{N+2}, t=T_{N+1}, and t=T_{N} may be referred to as “sampling timing”. The interval of the sampling timings may be a regular or different interval.

The information processing device 3 exemplarily obtains the amplitude change amount between the sampling timings based on the amplitude values (voltage values) that are obtained at the sampling timings. For example, the processor 31 of the information processing device 3 may obtain the differential between the amplitude values at neighboring sampling timings as the amplitude change amount between the sampling timings (this corresponds to process P13 in FIG. 8).

Exemplarily, the processor 31 may obtain the amplitude change amount between the sampling timing t=T_{N+2} and the next sampling timing t=T_{N+1} as the absolute value |A_{α+1}−A_{α+2}|. Similarly, the processor 31 may obtain the amplitude change amount between the sampling timing t=T_{N+1} and the next sampling timing t=T_{N} as the absolute value |A_{α}−A_{α+1}|.

The processor 31 repeatedly conducts such computation through the samplings per unit time, adds the obtained amplitude change amounts like |A_{α}−A_{α+1}|+|A_{α+1}−A_{α+2}|+ . . . , and may thereby calculate “extended wavelength” (this corresponds to process P14 in FIG. 8).

Note that as exemplified in FIG. 13, in a case where the electric wave sensor value is represented by the voltage value [V], the unit of “extended wavelength” may be represented by “voltage/time” (V/min), for example.

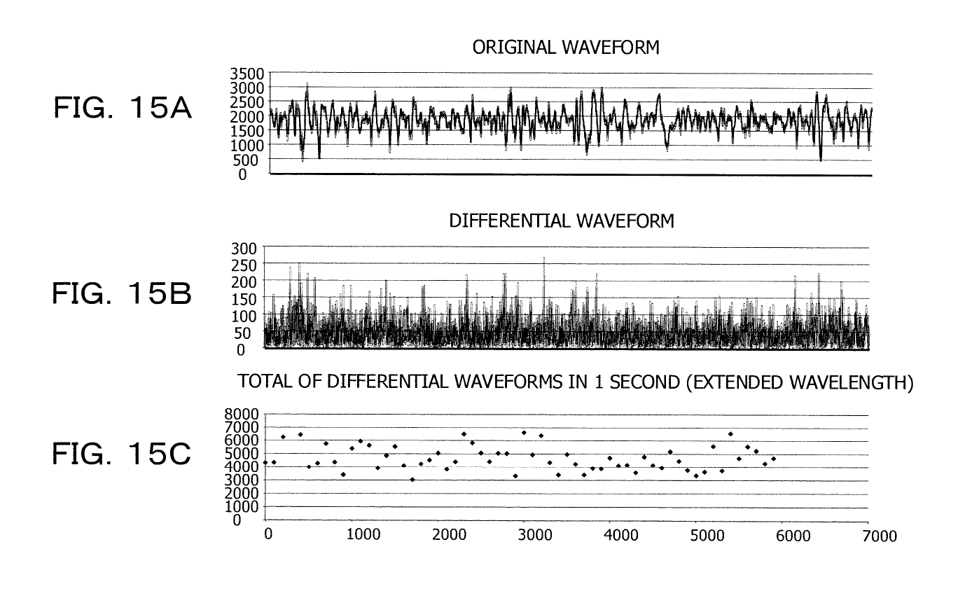

FIG. 15A to FIG. 15C illustrate one example of an extended wavelength calculation process. FIG. 15A represents an example of an original waveform of the electric wave sensor value, FIG. 15B represents one example of a differential waveform, and FIG. 15C represents one example of a value that results from totaling the differential waveform through a prescribed time (exemplarily, one second).

The differential waveform exemplified in FIG. 15B represents the amplitude change amount in each prescribed very-short time about the original waveform exemplified in FIG. 15A and is exemplarily a differential waveform that is calculated about the sampling period for 1 kHz. Consequently, the amplitude change amount exemplarily represents the amplitude change amount per 1/1000 second.

The differential waveform may be totaled every second in the time period ΔT that is exemplified in FIG. 13. For example, every 1000 amplitude change amounts may be totaled in the time period ΔT. Accordingly, as exemplified in FIG. 15C, the extended wavelength in the time period ΔT is calculated.

Note that in a case where the number of samplings of the amplitude value per unit time is too small, the calculation precision of “extended wavelength” lowers. In a case where the number of samplings is too many, the computation load becomes high, and a computation delay or the like may occur. Thus, the number of samplings may be set to a realistic range. In addition, “extended wavelength” may be averaged with respect to time for a prescribed time. For example, the average of 60 “extended wavelengths”, which are obtained in 1 minute while the unit time is set to 1 second, may be obtained.

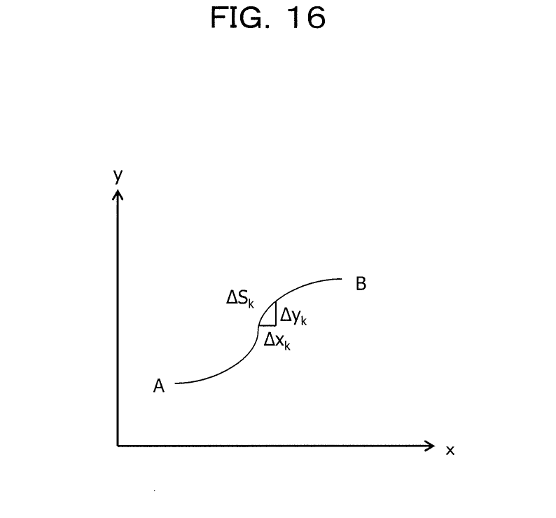

“Extended wavelength” may be calculated as follows. For example, FIG. 16 illustrates an example where “extended wavelength” of curve AB is calculated. The interval between A and B is divided into n very-short intervals, each of the very-short intervals is approximated by a line segment, and the sum Sn of the lengths is expressed by the following formula 1.

$$Sn = {\sum }_{k = 1}^{n} Δ {S}_{k} \qquad [ Formula 1 ]$$

Given that a very-small displacement of the very-short interval in the x direction is Δxk and a very-small displacement of the very-short interval in the y direction is Δyk, ΔSk is expressed by the following formula 2 by the Pythagorean theorem.

ΔSₖ=\sqrt{(Δxₖ)²+(Δyₖ)²} [Formula 2]

As expressed by the following formula 3, given that the number n of the very-short intervals of the formula 2 is increased to infinity, the sum Sn approaches the length L of curve AB.

$$L = {lim}_{n \to ∞} {S}_{n} = {lim}_{n \to ∞} {\sum }_{k = 1}^{n} Δ {x}_{k} = {lim}_{n \to ∞} {\sum }_{k = 1}^{n} \sqrt{{( Δ {x}_{k} )}^{2} + {( Δ {y}_{k} )}^{2}} \qquad [ Formula 3 ]$$

In the formula 3, supposing that the x direction is set as the time axis and the sampling period of the electric wave sensor value is a regular period (for example, 1 kHz), “xₖ” is a regular value, the electric wave sensor value (for example, the amplitude value) is substituted for “yₖ”, and “extended wavelength” is thereby calculated.

In processes P14 and P15 in FIG. 8, the information processing device 3 may calculate “body movement amount” of the user based on above-described “extended wavelength” and may assess the sleep state of the user based on calculated “body movement amount” (process P16).

Exemplarily, a computation formula (which may also be referred to as “criterion formula”) that is referred to as “AW2 formula” or “Cole formula” may be applied to the sleep assessment. For example, in a case where the value that is computed by “AW2 formula” or “Cole formula” based on “body movement amount” obtained in a certain assessment time (exemplarily, several minutes) is a certain threshold value or more, an assessment of “asleep” may be made. In a case where the computed value is less than the threshold value, an assessment of “awake” may be made.

Based on the sleep assessment result, the information processing device 3 may generate a control signal for controlling the operation of the air conditioner 7, dimming of the lighting equipment 8, or the like, for example, and may transmit the control signal to the air conditioner 7 or the lighting equipment 8 (process P17). Accordingly, the interior space may be controlled to a comfortable environment for the user.

Note that the result of the sleep assessment by the information processing device 3 may properly be output to the output apparatus such as a display or a printer (process P18). Further, the extended wavelength that is calculated in process P14 or the body movement amount that is calculated in process P15 may properly be output to the output apparatus such as a display or a printer. In this case, the situation of a calculation procedure of information used for the sleep assessment may be checked.

In addition, process P14 in which the extended wavelength is calculated may be optional as indicated by dotted lines in FIG. 8. For example, based on the amplitude change amount of the electric wave sensor value, which is calculated in process P13, the body movement amount may be calculated in process P15 without calculating the extended wavelength. This point may be the same in the second example (FIG. 18) and the third example (FIG. 19), which will be described later.

As described in the above, in the first example, the transmission power of the electric wave sensor 21 included in the sensor 2 is controlled in accordance with the gravity direction that is detected by the inertia sensor 22 included in the sensor 2. Thus, the detection sensitivity of the electric wave sensor 21 may be optimized in accordance with the installation place of the sensor 2.

Consequently, the separate sensors 2 do not have to be prepared for the installation places, and a switch or the like that switches the processes in accordance with the installation place does not have to be included in the sensor 2. Thus, the manufacturing cost or inventory cost for the sensor 2 may be reduced, and trouble such as adjustment work in installation of the sensor may be omitted.

Because trouble such as adjustment work in installation of the sensor may be omitted, in the first installation of the sensor, this allows the sensor 2 to be installed by the idea of the sensor installer without performance of detailed adjustment work by the installer.

In addition, because the installation place of the sensor 2 may automatically be recognized based on the inertia sensor value, for example, trouble for work such as addition or registration of the information, by which the installation place is identifiable, for the sensor 2 or the information processing device 3 may be omitted. Further, trouble for changing the analysis process or analysis algorithm of the sensor information in accordance with the installation place of the sensor 2 may be omitted.

Consequently, for example, the installation place of the sensor 2 may freely be changed after installation, and the degree of freedom or flexibility of initial settings of the sensor 2 or the change in layout of the interior space may be improved.

Further, because the detection sensitivity of the electric wave sensor 21 is optimized in accordance with the installation place of the sensor 2, occurrence of excess or deficiency in the detection sensitivity may be inhibited, and the possibility of occurrence of false detection in the body movement detection or a false assessment in the sleep assessment may be decreased. Consequently, the precision of the body movement detection or the sleep assessment of the user may be improved.

In addition, because the transmission power of the electric wave sensor 21 is optimized in accordance with the installation place, efficient power consumption of the sensor 2 may be intended.

Further, because the precision of the body movement detection or the sleep assessment of the user may be improved, for example, the precision of environment control of the interior space may be improved by using the result of the body movement detection or the sleep assessment, and efficient environment control may be intended.

For example, the information processing device 3 adaptively controls the operation of the air conditioner 7, dimming of the lighting equipment 8, or the like in accordance with the result of the body movement detection or the sleep assessment and may thereby control the interior space to a comfortable environment for the user. Consequently, the precision of the body movement detection or the sleep assessment of the user is improved, and it thereby becomes possible to intend the efficient environment control.

Note that the installation place categorization table 321 that is exemplified in FIG. 9 may be created based on angle information of the inertia sensor 22, which is calculated from the inertia sensor values, in other words, the angle information of the sensor 2.

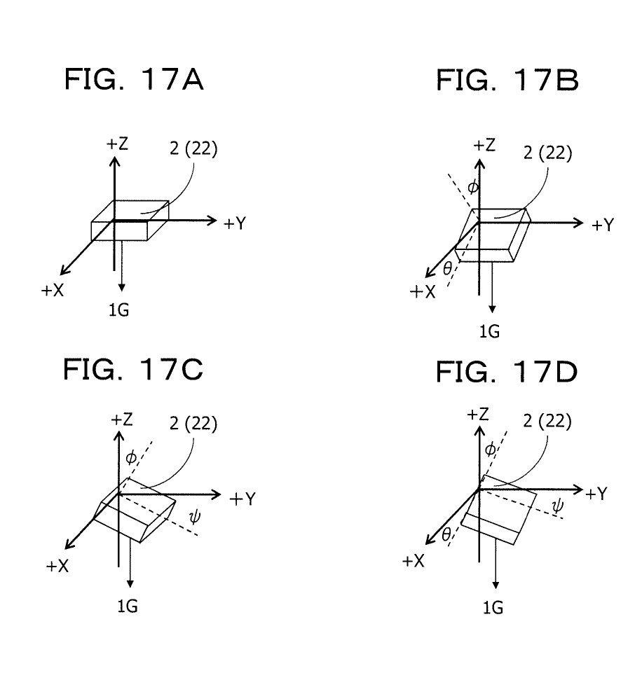

For example, as schematically exemplified in FIG. 17A to FIG. 17D, the angle information of the inertia sensor 22 may be obtained by the following formulas 4 to 6 based on the accelerations Ax, Ay, and Az that are respectively obtained with respect to the detection axes X, Y, and Z of the inertia sensor 22.

$$\begin{matrix}θ = {tan}^{- 1} ( \frac{Ax}{\sqrt{{A}_{y}^{2} + {A}_{z}^{2}}} ) & [ Formula 4 ] \\ ψ = {tan}^{- 1} ( \frac{{A}_{y}}{\sqrt{{A}_{x}^{2} + {A}_{z}^{2}}} ) & [ Formula 5 ] \\ φ = {tan}^{- 1} ( \frac{\sqrt{{A}_{x}^{2} + {A}_{y}^{2}}}{{A}_{z}} ) & [ Formula 6 ]\end{matrix}$$

The angle information obtained by the above formulas 4 to 6 is associated with the place where the sensor 2 is assumed to be installed in the interior space, and it is thereby possible to create the installation place categorization table 321.

Note that without using the installation place categorization table 321, the transmission power of the electric wave sensor 21 may be controlled based on the angle information obtained by the formulas 4 to 6.

### Second Example

Next, the second example will be described with reference to a flowchart exemplified in FIG. 18.

The second example is an action example of correcting the detection signal level of the electric wave sensor 21 in accordance with the gravity direction detected by the inertia sensor 22.

Note that the correction of the detection signal level of the electric wave sensor 21 may be considered as one example of changing the process about the reflected wave of the transmitted electric wave by the electric wave sensor 21.

The flowchart exemplified in FIG. 18 corresponds to the flowchart in which processes P12 and P23 in FIG. 8 of the first example are respectively replaced by processes P12a and P23a and process P24 in FIG. 8 is deleted.

Thus, processes P11, P13 to P17, P21, and P22 in FIG. 18 may respectively be the same as processes P11, P13 to P17, P21, and P22, which are already described with reference to FIG. 8.

In process P23a, the information processing device 3 may control the amplification factor of the electric wave sensor value in process P12a, for example, such that the electric wave sensor value has the appropriate detection signal level (detection sensitivity, in other words) corresponding to the installation place of the sensor 2 that is assessed based on the inertia sensor value.

For example, the information processing device 3 may decide the correction value of the amplification factor that corresponds to the installation place of the sensor 2. The amplification factor of the electric wave sensor value may exemplarily be made larger as the distance between the sensor 2 and the user becomes longer. Thus, the correction value of the amplification factor may be decided similarly to the relationship in the table 322 exemplified in FIG. 11.

For example, as for the correction value of the amplification factor, in a case where the reference correction value in the wall installation of the sensor 2 is expressed as “large”, the correction value in the ceiling installation is expressed as “intermediate”, and the correction value in the bed installation is expressed as “small”, the information processing device 3 may correct the amplification factor of the present electric wave sensor value by the correction value corresponding to each of the installation place.

Alternatively, the amplification factor of the electric wave sensor value may be corrected based on information such as a table in which the angle information obtained by the already-described formulas 4 to 6 is associated with the place where the sensor 2 is assumed to be installed in the interior space. Instead, the amplification factor of the electric wave sensor value may be corrected based on the angle information obtained by the formulas 4 to 6.

Note that the amplification factor to be corrected may be the amplification factor of the operational amplifier 215 in the electric wave sensor 21, which is exemplified in FIG. 4. In that case, the information processing device 3 may generate a control signal that includes the correction value of the amplification factor (which may be referred to as “amplification factor control signal”) and may transmit the control signal to the sensor 2 (for example, the processor 23).

Further, the target of the correction is not limited to the amplification factor but may be “extended wavelength” that is calculated in process P14 or may be “body movement amount” that is calculated in process P15 based on “extended wavelength”, as exemplified by dotted line arrows in FIG. 18.

Because both of “extended wavelength” and “body movement amount” are likely to become smaller as the distance between the sensor 2 and the user becomes longer, the detection sensitivity of the electric wave sensor 21 may be likely to become lower. Consequently, the correction values of both of “extended wavelength” and “body movement amount” may be decided in a similar relationship to the large-small relationship of the correction value for the amplification factor.

Note that correction of “extended wavelength” may exemplarily be realized by correcting the candidate amplitude change amount of the electric wave sensor value, which is added to “extended wavelength”. Thus, the computation amount by the processor 23 may be suppressed.

As described in the above, in the second example, the detection value of the electric wave sensor 21 (or information obtained based on the detection value) is corrected in accordance with the gravity direction detected by the inertia sensor 22. Thus, the detection sensitivity of the electric wave sensor 21 may be optimized in accordance with the installation place of the sensor 2.

Consequently, in addition to obtainment of work and effects that are equivalent to the first example, the second example is different from the first example, and the transmission power of the electric wave sensor 21 does not have to be controlled. Thus, a delay of the body movement detection or the sleep assessment, which accompanies the transmission power control, may be inhibited.

### Third Example

Next, the third example will be described with reference to a flowchart exemplified in FIG. 19.

The third example is an action example of correcting the threshold value of the body movement amount that is obtained based on the electric wave sensor value (or the sleep assessment based on the body movement amount), in accordance with the gravity direction detected by the inertia sensor 22. The correction of the threshold value may be considered as another example of changing the process about the reflected wave of the transmitted electric wave by the electric wave sensor 21.

The flowchart exemplified in FIG. 19 corresponds to the flowchart in which processes P15 and P23 in FIG. 8 of the first example are respectively replaced by processes P15b and P23b and process P24 in FIG. 8 is deleted.

Thus, processes P11 to P14, P16, P17, P21, and P22 in FIG. 19 may respectively be the same as processes P11 to P14, P16, P17, P21, and P22, which are already described with reference to FIG. 8.

In process P23b, the information processing device 3 may control the threshold value such that the threshold value to be used for an assessment about the body movement amount calculated in process P15b becomes the appropriate value (detection sensitivity, in other words) corresponding to the installation place of the sensor 2 that is assessed based on the inertia sensor value.

For example, the information processing device 3 may decide the assessment threshold value of the body movement amount that corresponds to the installation place of the sensor 2 that is assessed based on the inertia sensor value. Correction or decision of the assessment threshold value may also exemplarily be considered as controlling the reference value of the body movement amount that is dealt with as a noise component.

Because the body movement amount calculated in process P15b exemplarily tends to become smaller as the distance between the sensor 2 and the user becomes longer, the assessment threshold value of the body movement amount may be corrected so as to become small. Making the assessment threshold value smaller may be considered as making the detection sensitivity of the body movement amount higher.

For example, the assessment threshold value of the body movement amount may be expressed as “small” in the wall installation in which the distance between the sensor 2 and the user becomes relatively long in the interior space, as “intermediate” in the ceiling installation, and as “large” in the bed installation. Based on the large-small relationship, the information processing device 3 may decide or set the threshold value corresponding to each of the installation place as the assessment threshold value of the body movement amount.

Exemplarily, because the distance between the sensor 2 and the user is longer in the ceiling installation than the bed installation, the calculated body movement amount tends to become smaller.

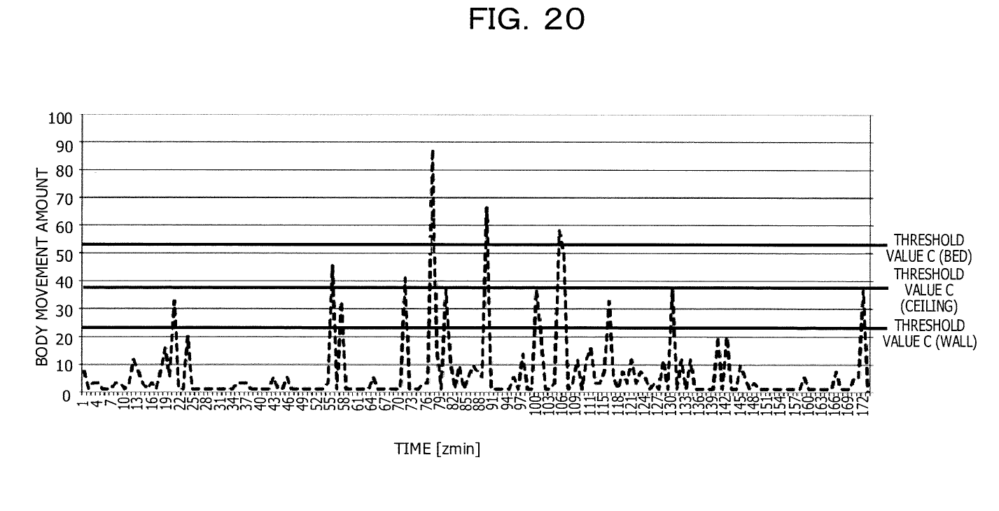

Consequently, in the ceiling installation, the information processing device 3 may correct or set an assessment threshold value C to a smaller value than the bed installation as schematically exemplified in FIG. 20, for example, and may make the detection sensitivity of the body movement amount higher.

Conversely, in the bed installation, the information processing device 3 may correct or set the assessment threshold value C to a larger value than the ceiling installation and may make the detection sensitivity of the body movement amount lower.

Note that the body movement amount calculated in the wall installation tends to become much smaller than the ceiling installation. Thus, in the wall installation, the information processing device 3 may correct or set the assessment threshold value C to a much smaller value than the ceiling installation.

Further, as exemplified by dotted line arrows in FIG. 19, the threshold value that is corrected in accordance with the installation place of the sensor 2 may be the threshold value (which may also be referred to as reference value) about the signal level that is dealt with as a noise component in process P12 and does not have to be processed. The threshold value that is used for the sleep assessment in process P16 may be corrected in accordance with the installation place of the sensor 2.

Similarly to the assessment threshold value of the body movement amount, the threshold value of the signal level or the threshold value used for the sleep assessment may be controlled to a smaller value as the distance between the sensor 2 and the user becomes longer, in order to make the detection sensitivity higher.

Consequently, as exemplified in FIG. 20, for example, the information processing device 3 may control the threshold value of the signal level or the threshold value of the sleep assessment in accordance with the installation place of the sensor 2, based on the large-small relationship that expresses the wall installation as “small”, the ceiling installation as “intermediate”, and the bed installation as “large”.

Alternatively, the assessment threshold value may be corrected based on information such as a table in which the angle information obtained by the already-described formulas 4 to 6 is associated with the place where the sensor 2 is assumed to be installed in the interior space. Instead, the assessment threshold value may be corrected based on the angle information obtained by the formulas 4 to 6.

As described in the above, the threshold value used for the body movement detection or the sleep assessment based on the detection value of the electric wave sensor 21 (or information obtained based on the detection value) is corrected in accordance with the gravity direction detected by the inertia sensor 22. Thus, the detection sensitivity of the electric wave sensor 21 may be optimized in accordance with the installation place of the sensor 2.

Consequently, in addition to obtainment of work and effects that are equivalent to the first example, the third example is similar to the second example, and the transmission power of the electric wave sensor 21 does not have to be controlled. Thus, a delay of the body movement detection or the sleep assessment, which accompanies the transmission power control, may be inhibited.

(Other Matters)

Note that the above-described first example may be conducted while being combined with either one or both of the second example and the third example. In other words, the transmission power control of the electric wave sensor 21 that corresponds to the installation place of the sensor 2 and control of the process about the reflected wave of the transmitted electric wave by the electric wave sensor 21 may be conducted in combination.

Because the combined conduction enables the control of the detection sensitivity of the electric wave sensor 21 to be distributed into the transmission power control and the control of the process about the reflected wave, the control amounts by the individual pieces of control (in other words, a variable width) may be made smaller than a single piece of control. Consequently, a decrease in the control load may be intended, and lightening of the demanded performance of the sensor 2 or the information processing device 3 may be intended.

Further, in the embodiment that includes the above-described examples, descriptions are made about the modes in which the processes exemplified in FIG. 8, FIG. 18, and FIG. 19 is conducted by the information processing device 3 (for example, the processor 31). However, a portion or all of the processes exemplified in FIG. 8, FIG. 18, and FIG. 19 may be conducted by the sensor 2 (for example, the processor 23). The program corresponding to the process conducted by the sensor 2 may be stored in the memory 24 (see FIG. 5) of the sensor 2, for example.

For example, assessment process P22 exemplified in FIG. 8, FIG. 18, and FIG. 19 may be conducted by the processor 23 of the sensor 2. The processor 23 may transmit the information that indicates the installation place of the sensor 2, which is assessed based on the gravity direction detected by the inertia sensor 2, from the communication IF 25 (see FIG. 5) as one example of the communication portion to the information processing device 3, for example.

The information that indicates the installation place of the sensor 2 may exemplarily be information that indicates the difference among whether the sensor 2 is arranged in the lower portion (for example, the bed 5) of the space, whether the sensor 2 is arranged in the upper portion (for example, the ceiling) of the space, and whether the sensor 2 is arranged in the lateral portion (for example, the wall) of the space.

Based on the information that is acquired from the sensor 2, as already described in the first example, the information processing device 3 (for example, the processor 31) may control the transmission power of the electric wave sensor 21 in accordance with the installation place of the sensor 2.

In a mode in which the information processing device 3 conducts the calculation process, the correction process, and the sleep assessment process, for example, alternation of a program or data, which are read by the processor 31 of the information processing device 3 for an action, easily enables addition of functions or update of the information processing device 3. Consequently, without addition of alternation or the like to the sensor 2, alternation of the information processing device 3 easily enables centralized update or the like of the sensor system 1.

Further, in the embodiment that includes the above-described examples, the sleep assessment of the user in the interior space is described. However, an assessment about whether the user stays in the interior space or is absent may be performed based on the electric sensor value. The information processing device 3 may adaptively perform remote control of an action of the air conditioner 7 or the lighting equipment 8 in accordance with the staying or absence of the user.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to a showing of the superiority and inferiority of the invention. Although the embodiment of the present invention has been described in detail, it should be understood that the various changes, substitutions, and alterations could be made hereto without departing from the spirit and scope of the invention.

What is claimed is:

1. A sensor device comprising:
an inertia sensor;
an electric wave sensor; and
a controller configured to control transmission power of an electric wave of the electric wave sensor in accordance with a gravity direction which is detected by the inertia sensor.

2. The sensor device according to claim 1, wherein the controller performs, in a first case where the gravity direction is on the same side as a transmission side of the electric wave from the sensor device, control to make a transmission power of the electric wave in the first case higher than a transmission power of the electric wave in a second case where the gravity direction is on an opposite side to the transmission side of the electric wave from the sensor device.

3. The sensor device according to claim 1, wherein the controller performs, in a second case where the gravity direction is on an opposite side to a transmission side of the electric wave from the sensor device, control to make a transmission power of the electric wave in the second case lower than a transmission power of the electric wave in a second case where the gravity direction is on the same side as the transmission side of the electric wave from the sensor device.

4. The sensor device according to claim 1, wherein the controller performs, when the gravity direction is in a third case which is not a first case where the gravity direction is on the same side as a transmission side of the electric wave from the sensor device and a second case where the gravity direction is on the opposite side to the transmission side of the electric wave from the sensor device, control to make a transmission power of the electric wave in the third case higher than a transmission power of the electric wave in the first case.

5. The sensor device according to claim 2, wherein the sensor device is arranged in an upper portion of a space in a state where the transmission side of the electric wave is directed in a downward direction of the space.

6. The sensor device according to claim 5, wherein the sensor device is arranged in a ceiling of the space of an indoor place.

7. The sensor device according to claim 3, wherein the sensor device is arranged in a lower portion of a space in a state where the transmission side of the electric wave is directed in an upward direction of the space.

8. The sensor device according to claim 7, wherein the sensor device is arranged in a bed of the space of an indoor place.

9. The sensor device according to claim 4, wherein the sensor device is arranged in a lateral portion of a space in a state where the transmission side of the electric wave is directed in a lateral direction of the space.

10. The sensor device according to claim 9, wherein the sensor device is arranged in a wall of the space of an indoor place.

11. The sensor device according to claim 1, wherein the electric wave sensor is an electric wave sensor that is used for detection of body movement.

12. A sensor device comprising:
an inertia sensor;
an electric wave sensor;
a communication portion; and
a controller configured to cause the communication portion to transmit information that indicates whether the sensor device is arranged in a lower portion of a space or is arranged in an upper portion of the space in accordance with a gravity direction which is detected by the inertia sensor.

13. The sensor device according to claim 12, wherein the controller to control transmission power of an electric wave of the electric wave sensor in accordance with the gravity direction which is detected by the inertia sensor.

14. The sensor device according to claim 13, wherein the controller performs, in a first case where the gravity direction is on the same side as a transmission side of the electric wave from the sensor device, control to make a transmission power of the electric wave in the first case higher than a transmission power of the electric wave in a second case where the gravity direction is on an opposite side to the transmission side of the electric wave from the sensor device.

15. The sensor device according to claim 13, wherein the controller performs, in a second case where the gravity direction is on an opposite side to a transmission side of the electric wave from the sensor device, control to make a transmission power of the electric wave in the second case lower than a transmission power of the electric wave in a second case where the gravity direction is on the same side as the transmission side of the electric wave from the sensor device.

16. A sensor control device comprising:
a memory;
a communication portion; and
a processor coupled to the memory and the communication portion and configured to cause the communication portion to transmit a signal which controls transmission power of an electric wave transmitted by an electric wave sensor based on detection information of an inertia sensor which is received from a sensor device including the inertia sensor and the electric wave sensor, to the sensor device.

17. The sensor control device according to claim 16, wherein the detection information includes a measurement value that is measured by the inertia sensor or information that indicates a gravity direction which is detected by the inertia sensor.

18. The sensor control device according to claim 16, wherein the controller changes a process about a reflected wave of the transmitted electric wave based on the detection information.

19. The sensor control device according to claim 16, wherein the change in the process includes controlling a reference value of a signal level that corresponds to the reflected wave which is dealt with as a noise component.

20. The sensor control device according to claim 16, wherein the change in the process includes correcting a signal level that corresponds to the reflected wave.
